(19) United States
(12) Patent Application Publication
(10) Pub. No.: US 2004/0115801 A1
(43) Pub. Date: Jun. 17, 2004

(54) Model epithelial cell cultures

(75) Inventors: Yi-Chan James Lin, Edmonton (CA); Yun Kau Tam, Yuen Long (HK); Hugh Alexander Semple, Edmonton (CA); Brian Duff Sloley, Edmonton (CA)

Correspondence Address: Albert Wai-Kit Chan, Law Office of Albert Wai-Kit Chan, World Plaza, Suite 604, 141-07 20th Avenue, Whitestone, NY, 11357, US

(73) Assignee: KINETANA GROUP INC.

(21) Appl. No.: 10/648,081

(22) Filed: Aug. 25, 2003

Related U.S. Application Data
(60) Provisional application No. 60/405,525, filed on Aug. 23, 2002.

(51) Int. Cl. C12N 5/06; C12N 5/02

(52) U.S. Cl. 435/325; 435/404

(57) ABSTRACT

In one aspect, the invention provides cell cultures and culture conditions that facilitate the establishment of an intestinal epithelial cell culture that has characteristics of the intestinal epithelium, such as: transepithelial electrical resistance, enzyme expression and microvilli morphology. Cell cultures of the invention may use fatty acids such as sodium butyrate and kinase modulators such as 2-{1-[3-(amidinothio)propyl]-1H-indol-3-yl}-3-(1-meethylindol-3-yl)-maleimide methanesulfonate.

This application claims benefit of U.S. Serial No. 60/405,525, filed Aug. 23, 2002, the content of which is incorporated into this application by reference.

Throughout this invention, various references are referred to. Disclosures of these publications in their entireties are hereby incorporated by reference into this application to more fully describe the state of the art to which this invention pertains.

### FIELD OF THE INVENTION

The invention is in the field of epithelial cell cultures and cell culture techniques, particularly model intestinal epithelia.

### BACKGROUND OF THE INVENTION

The intestinal epithelium plays an important role in disease, in part through its role in drug transport and absorption. In vitro cell culture systems may therefore be useful to model the behavior of the intestinal epithelium. However, epithelial cells grown in vitro do not necessarily mature or differentiate into cells or tissues that exhibit the characteristics of the intestinal epithelium in vivo. Intestinal epithelial cells express intestinal enzymes, exhibit long and dense microvilli, and exhibit low monolayer transepithelial electrical resistance (TEER). These characteristics in combination may help to determine in vivo drug and nutrient absorption kinetics (Eckert and Randall 1983).

A variety of human intestinal cell lines have been described, including Intestine 407, FHs 74 Int, Hs 1. Int, Hs 738. St/Int, CaCo-2, and HT29 (ATCC catalog, Quaroni and Hochman 1996). Intestine 407, CaCo-2, and HT29 are thought to be derived from colorectal tumors and to be tumorigenic. Such cells may for example be used in assays to assess pharmacokinetic characteristics of natural products (J. A. Ringer, 2001). FHs 74 Int, Hs 1 Int, and Hs 738 St/Int are thought to be derived from unknown regions of fetal gastrointestinal tract (ATCC catalog, Owens et al. 1976), and to exhibit fibroblast morphology. FHs 74 Int is reportedly nontumorigenic. Another reportedly nontumorigenic cell line, obtained from normal human adult tissue, is known as SCBN (Pang et al. 1996, Teoh et al. 2000, Engman et al. 2001, O'Loughlin et al. 2001). Pang et al. (1996) describes the establishment of SCBN cells from a duodenal biopsy of a patient (B.N.), who had no evidence of inflammation, neoplasia, or abnormality. Teoh et al. (2000) describes the use of SCBN cells in transwell setups for testing the effects of Giardia on actin cytoskeleton and monolayer transepithelial electrical resistance (TEER). O'Loughlin et al. (2001) describes the effects of interleukin 2 on ion secretion and cell proliferation of SCBN cells.

When SCBN cells were grown in culture conditions of DMEM medium supplemented with 10% fetal bovine serum, 1× Non-essential amino acids, and 10 mM HEPES as the culture media (Pang et al. 1996, Teoh et al. 2000, Engman et al. 2001, O'Loughlin et al. 2001), the cells were observed to exhibit tight junction formation in monolayer after reaching confluence. However, the cells were in a dedifferentiated, or undifferentiated state as are indicated by the short and sparse microvilli, and lack of brush border enzyme expression, e.g. alkaline phosphatase and sucrase isomaltase. The transepithelial electrical resistance (TEER) for the confluent SCBN monlolayers cultured by the above-mentioned condition exceeds 1000 ohm.cm² for 3 day cultures in the lab and 2000 ohm.cm² for 14 day cultures (Engman et al. 2001), whereas TEER values for human intestine vary from 12-69 ohm.cm² (Madara and Trier, 1987). Engman et al. (2001) also examined the presence of P450 in SCBN cells and concluded, inter alia, that SCBN cells were not suitable for the study of drug transport.

Several cell lines, including CaCo-2, HT29, and MDCK, have been used as permeability screening tools in drug discovery. Of these cells lines, CaCo-2 has been used extensively. Under tissue culture conditions, CaCo-2 cells reportedly differentiate spontaneously after cultures reach confluence (Quaroni and Hochman 1996). Although CaCo-2 cells are derived from colorectal cancers, differentiated CaCo-2 cells show characteristics of small intestinal epithelial cells, including brush border microvilli, tight junctions, and certain levels of small intestinal enzyme expression (Quaroni and Hochman 1996). However, TEER values from CaCo-2 monolayers reportedly vary with a range from 360 ohm.cm² to 2400 ohm.cm² (Hilgendorf et al 2000, Pontier et al. 2001).

Since the in vivo human small intestine have low TEER values, express brush border enzymes, long microvilli, and transporter activity (Madara and Trier, 1987), it is believed that an ideal in vitro cultured cell monolayer for drug screening should mimic the above property. The current invention intends to generate an in vitro cell monolayer with low TEER values, expression of brush border enzymes, long microvilli, and transporter activity with a nontumorigenic human intestinal cell line.

### SUMMARY OF THE INVENTION

In one aspect, it has surprisingly been found that cell culture conditions may be provided that facilitates the establishment of an intestinal epithelial cell culture that has many of the characteristics of the intestinal epithelium in vivo.

In one aspect of the invention, a method of culturing cells is provided which involves growing intestinal epithelial cells to form a confluent cell layer in a culture media. The intestinal epithelial cells may express an enzyme activity characteristic of differentiated intestinal epithelia. For example, measurable enzyme activity may be found for alkaline phosphatase, dipeptidyl peptidase IV, aminopeptidase or amino peptidase A.

Culture media for use in various aspects of the invention may include an amount of a differentiating compound effective to maintain a reduced transepithelial electrical resistance of the confluent cell layer. The differentiating compound may for example be a fatty acid, such as a butyrate salt (including sodium butyrate). Sodium butyrate has been used in inducing cell differentiation in varied cell lines, including colorectal tumor cell lines, CaCo-2 and HT29. The effect of sodium butyrate on CaCo-2 and HT29 includes the induction of cell cycle arrest, differentiation and apoptosis, increased expression of digestive enzymes, and increased TEER (Mariadason et al 2000, Wang et al. 2001). In alternative embodiments, the effective amount of the differentiating compound may for example be greater than 1 mM, from about 1 mM to about 10 mM or about 5 mM.

Culture media for use in various aspects of the invention may also include an amount of a kinase modulator effective in combination with the differentiating compound to support growth of long microvilli, such as microvilli longer than 1 micrometer, on a portion of the epithelial cells. The kinase modulator may be a kinase inhibitor. Alternatively, the kinase modulator may have an activity such as activating JNK1, inhibiting GRK-5, inhibiting PKC, inhibiting MAPKAP kinase-1beta and inhibiting p70 S6 kinase. In some embodiments, the kinase modulator may be 2-{1-[3-(amidinothio)propyl]-1H-indol-3-yl}-3-(1-meethylindol-3-yl)-maleimide methanesulfonate (which may be available commercially from Sigma RBI, St. Louis, Mo., USA as Ro 31-8220 under catalogue number R-136; see also Moore, J. B. et al. 1998 and Beltman, J. et al. 1996). In alternative embodiments, the effective amount of the kinase modulator may for example be greater than 1 micromolar, from about 1 micromolar to about 10 micromolar, or about 5 micromolar. The structure of Ro 31-8220 is given in the product monographs as follows:

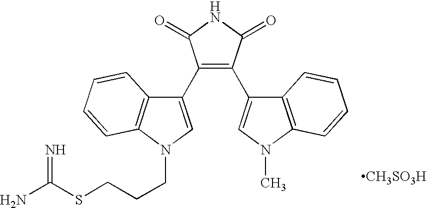

In one aspect of the invention, a confluent cell layer may be grown on an extracellular matrix base, which may be a collagen (such as rat-tail collagen). The culture media may be essentially free of animal serum, and may include nutrients such as D-glucose, L-glutamine, sucrose, pyruvic acid, a pH buffer and one or more amino acids. The culture may be maintained at an optimal temperature for the cells, such as a temperature of about 36 degrees Celsius to 38 degrees Celsius for mammalian cells.

Cell lines for use in various aspects of the invention may for example be nontumorigenic intestinal epithelial cells, such as human cells derived from a duodenal biopsy, as was the case for SCBN cells.

In alternative embodiments, the transepithelial electrical resistance of the confluent cell layer may be less than about: 200, 150, 100 or 50 ohm/cm², any value within the range of between 50 and 200 ohm/cm², or 60-200 ohm/cm²

In alternative embodiments, the proportion intestinal epithelial cells having microvilli longer than 1 micrometer may be greater than: 5%.

In an alternative aspect, the invention provides methods of processing mixtures such as aqueous compositions made up of a plurality of compounds, such as therapeutically active compounds mixed with inactive compounds, to obtain a physiologically modified composition. Such methods may include applying the mixture or aqueous composition to a first side of an in vitro confluent cell layer, such as an intestinal epithelial monolayer, and extracting the modified composition from a second side of the cell layer.

In one aspect the invention provides a Ginkgo extract processed in accordance with the methods of the invention, which may for example have an application to the separation and identification of absorbable ingredients from an herb or herbal extract which are responsible for the preparation's efficacy. For instance, an ingredient profile for a commercial extract differs from the profile of the same ingredients after passing across the cell monolayers of the invention. This profile of ingredients which passed across the cell membrane may correlate with the absorption profile of these ingredients in humans in vivo and thus with its effectiveness.

### BRIEF DESCRIPTION OF THE DRAWINGS

FIG. 1: illustrates the TEER values of SCBN cells in different culture conditions. TEER measurement was conducted by using EVOM voltmeter from World Precision Instrument. As shown, the culture conditions were: DMEM supplemented with fetal bovine serum (FBS); base medium; base medium supplemented with 5 μM of Ro31-8220; base medium supplemented with 5 mM of Sodium butyrate; and base medium supplemented with 5 μM of Ro31-8220 and 5 mM of sodium butyrate.

FIG. 2: illustrates the expression of enzymes in SCBN cells cultured under culture conditions of the invention. Enzyme activity is expressed as milliunit per mg of protein. One unit equals the digestion of one μmole of substrate per minute at 37° C. AP: Alkaline phosphatase; DPP: dipeptidylpeptidase-IV; APN: aminopeptidase N; APA: aminopeptidase A.

FIG. 3: Polyethylene glycol isomer permeabilities in the invention and CaCo-2 monolayers.

FIG. 4: Paracellular marker and PEG permeabilities in the invention and CaCo-2 monolayers and human in vivo.

FIG. 5: Human in vivo Fₐ vs. P_{eff} of 35 compounds in the invention, 21-day CaCo-2 and human in vivo jejunal (Sun, Lennernas et al. 2002)

FIG. 6: The basis for a surface area correction of permeability. From Wilson, TH: Intestinal Absorption, WB Saunders Co., Philadelphia, 1962, as referenced in Shargel & Yu: Applied Pharmacokinetics and Biopharmaceutics, 3rd Edition, Appleton & Lange, Norwalk, Conn., 1993.

FIG. 7: B:A/A:B ratio across the monolayers of the invention for efflux pump substrates etoposide, verapamil, quinidine and vinblastine. The invention shows efflux activity.

FIG. 8. Representative chromatogram of intact flavonol glycosides determined in fluid from the apical side of the cell layer 60 minutes after application of standardized ginkgo extract (equivalent of 10 mg extract).

FIG. 9. Representative chromatogram of intact flavonol glycosides determined in fluid from the basal side of the cell layer 60 minutes after application of standardized ginkgo extract (equivalent of 10 mg extract).

FIG. 10. Representative chromatogram of ginkgolides determined in fluid from the apical side of the cell membrane 60 minutes after application of standardized ginkgo extract (equivalent of 20 mg extract).

FIG. 11. Representative chromatogram of ginkgolides determined in fluid from the basal side of the cell layer 60 minutes after application of standardized ginkgo extract (equivalent of 400 mg extract).

### DETAILED DESCRIPTION OF THE INVENTION

In one aspect, the invention relates to uses for confluent cell layers. In some embodiments, these cell layers may be monolayers, in the sense that they are composed primarily of a single cellular layer. In alternative embodiments, it is possible that more than one cellular layer may be present, for example to provide nutritive or physical support for an active cellular layer that models a tissue such as the intestinal epithelium.

In one aspect of the invention, cells for use in the invention may be characterized by enzyme expression patterns. These patterns may be chosen as indicative that such cells are in a cellular state suitable for use in the invention. For example, marker enzymes may be selected from the wide range of enzymes that typically characterize differentiated cells of a desired cell type. For example, in embodiments of the invention relating to the use of intestinal epithelial cells, measurable enzyme activity may be found in alternative embodiments for alkaline phosphatase, dipeptidyl peptidase IV, aminopeptidase or amino peptidase A. The selected enzymes may of course be varied based on knowledge of which enzymes are active in a desired cell type.

In one aspect of the invention, culture media for use in various aspects of the invention may include an amount of a differentiating compound. A “differentiating compound” is a compound which either alone or in combination with other components of the media achieves a selected effect on the cells relating to their state of differentiation. For example, a differentiating compound may be effective to maintain a reduced transepithelial electrical resistance of a confluent cell layer. In some embodiments, particularly for use with human intestinal epithelia, the differentiating compound may for example be a fatty acid, such as a butyrate salt (including sodium butyrate). Sodium butyrate has been used in inducing cell differentiation in varied cell lines, including colorectal tumor cell lines, CaCo-2 and HT29. The effect of sodium butyrate on CaCo-2 and HT29 includes the induction of cell cycle arrest, differentiation and apoptosis, increased expression of digestive enzymes, and increased TEER (Mariadason et al 2000, Wang et al. 2001). In alternative embodiments, the effective amount of the differentiating compound may for example be greater than 1 mM, from about 1 mM to about 10 mM or about 5 mM. The appropriate concentration of differentiating compound for alternative embodiments may be determined empirically.

Culture media for use in various aspects of the invention may also include an amount of a kinase modulator. Kinase modulators are compounds that change the level of activity of a kinase in the cell culture. Such compounds may be effective alone or in combination with other compounds such as the differentiating compound. The physiological effect of the kinase modulator may be to facilitate the differentiation of the cell culture. For example, in intestinal epithelial cell culture, the kinase modulator may support growth of long microvilli, such as microvilli longer than 1 micrometer, on a portion of the epithelial cells. In alternative embodiments, the proportion of intestinal epithelial cells having long microvilli may be greater than: 5%, 10%, 15%, 20%, 25%, 30%, or any value between 5% and 30% which includes but is not limited to 6, 7, 8, 9, 10%. In another embodiment, it is between 5-20%. “Long microvilli” may for example mean microvilli longer than 1 micrometer, particularly for mammalian intestinal epithelial cells.

The kinase modulator may be a kinase inhibitor or activator. For example, particularly for intestinal epithelial cells, the kinase modulator may have an activity such as activating JNK1, inhibiting GRK-5, inhibiting PKC, inhibiting MAPKAP kinase-1beta and inhibiting p70 S6 kinase. In some embodiments, the kinase modulator may be 2-{1-[3-(amidinothio)propyl]-1H-indol-3-yl}-3-(1-meethylindol-3-yl)-maleimide methanesulfonate (which may be available commercially from Sigma RBI, St. Louis, Mo., USA as Ro 31-8220 under catalogue number R-136; see also Moore, J. B. et al. 1998 and Beltman, J. et al. 1996). In alternative embodiments, the effective amount of the kinase modulator may for example be greater than 1 micromolar, from about 1 micromolar to about 10 micromolar, or about 5 micromolar. The appropriate concentration of kinase modulator for use in alternative embodiments may be determined empirically.

In one aspect of the invention, a confluent cell layer may be grown on an extracellular matrix base, which may be a collagen (such as rat-tail collagen). Other compositions, artificial and biological are know to support growth of confluent cell layers, providing nutritive as well as physical support, such as extracellular matrix from EHS cells.

In some embodiments, the culture media may be essentially free of animal serum, and may include nutrients such as D-glucose, L-glutamine, sucrose, pyruvic acid, a pH buffer and one or more amino acids. Other ingredients may be selected from the components of known media, such as: CaCl₂; CuSO₄; ; Fe(NO₃)₃; FeSO₄; MgSO₄; KCl; NaHCO₃; NaCl; Na₂HPO₄; NaH₂PO₄; ZnSO₄; L-Alanine; L-Arginine; L-Asparagine; L-Aspartic Acid; L-Cysteine; L-Cystine; L-Glutamic Acid; L-Glutamine; Glycine; L-Histidine; L-Isoleucine; L-Leucine; L-Lysine; L-Methionine; L-Phenylalanine; L-Proline; L-Serine; L-Threonine; L-Tryptophan; L-Tyrosine; L-Valine; D-Biotin; Choline Chloride; Folic Acid; myo-Inositol; Nicotinamide; D-Pantothenic Acid; Pyridoxine; Riboflavin; Thiamine; Vitamin B-12; HEPES; Hypoxanthine; Methyl Linoleate; Phenol Red,; Sodium Salt; Putrescine; Sodium Pyruvate; DL-Thioctic Acid; or Thymidine. Cultures may be maintained at an optimal temperature for the cells, such as a temperature of about 36 degrees Celsius to 38 degrees Celsius for mammalian cells.

Cell lines for use in various aspects of the invention may for example be nontumorigenic intestinal epithelial cells, such as human cells derived from a duodenal biopsy, as was the case for SCBN cells.

In alternative embodiments, the transepithelial electrical resistance of the confluent cell layer may be less than about: 200 ohm.cm², 150 ohm.cm², 100 ohm.cm², 50 ohm.cm², or any value within the range of 50 and 200, or 60 and 200 ohm./cm². TEER values may be assayed using any conventional equipment capable of measuring electrical resistance, such as EVOH from World Precision Instruments.

In an alternative aspect, the invention provides methods of processing mixtures such as aqueous compositions made up of a plurality of compounds, such as therapeutically active compounds mixed with inactive compounds, to obtain a physiologically modified composition. Such methods may include applying an aqueous mixture which includes substances that are synthetic or natural in origin, to a first side of an in vitro confluent cell layer, such as an intestinal epithelial monolayer, and extracting the modified composition from a second side of the cell layer.

In one aspect, the invention provides materials and methods for processing compositions made up of a plurality of compounds, to obtain a physiologically modified composition. The compositions to be processed by the methods of the invention may for example include therapeutically active plant extracts. For processing, such compositions will generally be provided in an aqueous form.

In this aspect of this invention a composition may be applied to a first side of an in vitro confluent intestinal epithelial cell layer; and, a modified physiologically modified composition may be extracted from a second side of the confluent intestinal epithelial cell layer. By “physiologically modified” it is meant that the component parts of the initial composition are different either in relative amount or chemical identity from the component parts of the physiologically modified composition which includes components that have passed through the intestinal epithelial cell layer.

In various embodiments, the intestinal epithelial cell layer may be characterized by one or more of the following characteristics:

a) cells of the confluent intestinal epithelial cell layer express an enzyme activity selected from the group consisting of alkaline phosphatase, dipeptidyl peptidase IV, aminopeptidase and amino peptidase A; or

b) the confluent intestinal epithelial cell layer has a transepithelial electrical resistance less than about 200 ohm.cm²; or,

c) a portion of the cells of the confluent intestinal epithelial cell layer have microvilli longer than 1 micrometer.

In some embodiments, compositions for processing in accordance with the methods of the invention may include natural substances, such as crude extracts or formulations from natural products such as plants, which may be dissolved or suspended in an aqueous solution, such as a pH buffered solution or an artificial intestinal fluid. In alternative embodiments, a sample of these substances may be treated with artificial gastric juices to test prior to processing with the cell layers of the invention, for example to test for potential decomposition of ingredients under acidic conditions. In some embodiments, a known concentration of the solution or composition for processing, in either buffered solution or artificial intestinal fluid, may be applied to the apical or first side of the cell layer (membrane). Subsequently, one or more samples may be collected from the basal or second side of the cell layer, which may for example contain a cell culture medium or human plasma or components of human plasma such as albumin. In alternative embodiments, samples may be taken from the apical and basal sides for ingredient identification and/or quantification. Common analytical approaches such as high performance liquid chromatography (HPLC), mass spectrometry (MS), gas chromatography (GC), LC/MS, or GC/MS may then be used for the identification or quantification of the components present in the physiologically modified composition.

In some embodiments, aspects of the processing steps of the invention may be used in natural product development, for example to identify unknown active moieties in natural product mixtures; or to characterize the pharmacokinetics of active ingredients, for example the degree to which particular moieties are absorbable. In one aspect of the invention, active ingredients identified or characterized in this way may be formulated in therapeutic, insecticidal or pesticidal compositions in which the amount of absorbable ingredients is known. For example, if insect cells are used to provide cellular layers of the invention to model insect intestinal epithelia, the processing methods of the invention may be used to identify active insecticidal compounds or compositions found in natural mixtures known to have insecticidal activity. The processing methods of the invention may also be used to evaluate the quality or characteristics of formulated natural products.

This invention also provides a profile generated by the physiologically modified composition. In an embodiment, the profile is indicative that the composition is therapeutic.

This invention also provides a method to determine the batch variation of natural product comprising steps of generating a characteristic profile, sometimes, known as fingerprint of the natural product, and comparing the fingerprint of each batch of the product to determine whether said batch would be therapeutically useful.

This invention further provides a method to identify absorbable active ingredient by further purification of the ingredients identified in the profile. Standard purification procedures are known in the art. This invention also provides an absorbable active ingredient identified by the above method. This invention further provides a composition comprising an absorbable active ingredient. This composition may be a pharmaceutical composition which comprises an absorbable active ingredient and a pharmaceutically acceptable carrier.

For the purposes of this invention, “pharmaceutically acceptable carriers” mean any of the standard pharmaceutical carriers. Examples of suitable carriers are well known in the art and may include, but are not limited to, any of the standard pharmaceutical carriers such as a phosphate buffered saline solution and various wetting agents. Other carriers may include additives used in tablets, granules and capsules, etc. Typically such carriers contain excipients such as starch, milk, sugar, certain types of clay, gelatin, stearic acid or salts thereof, magnesium or calcium stearate, talc, vegetable fats or oils, gum, glycols or other known excipients. Such carriers may also include flavor and color additives or other ingredients. Compositions comprising such carriers are formulated by well-known conventional methods.

Finally, this invention provides a method for treating a subject comprising administering an effective amount of the pharmaceutical composition to said subject.

This invention also provides the uses of the absorbable active ingredient as a medicament for treatment of subjects.

In connection with the processing methods of the invention, various apparatus may be used, which are adapted to support an intestinal epithelial cell layer and to enable the fluid handling of solutions on either side of the cell layer. For example, U.S. Pat. No. 6,022,733 (issued to Tam, et al. on Feb. 8, 2000, and incorporated herein by reference) discloses an apparatus that may be adapted for this purpose. Alternative apparatus are also for example discussed in many of the documents cited in U.S. Pat. No. 6,022,733 (all of which are incorporated herein by reference).

### Examples

As illustrated in the following examples, alternative embodiments of the invention may be adapted to provide human intestinal epithelial cultures having characteristics of the natural human intestinal epithelium. In one embodiment, SCBN cells are seeded at confluent cell density (1E5 cells/well, i.e., 3.3E5 cells/cm²) with DMEM media supplemented with 10% fetal bovine serum (FBS) on rat-tail collagen coated plastic wares and secondly, 24 hours after seeding, culture media are changed with a combination of M199, DMEM, RPMI1640 media (v:v:v=4:3:3), supplemented with D-glucose (4.5 g/L), L-glutamine (4 mM), sucrose (5 mM), pyruvic acid (4 mM), ITS 1×, 1% Non-essential amino acids, 5 μM Ro-31-8220, and 5 mM sodium butyrate and cultured for another 48 hours. Under such a cell culture condition, SCBN cells have been found to produce long microvilli, express enzymes, and have the TEER value around 120 ohm.cm², so as to resemble intestinal epithelium cells in vivo.

In one aspect of the invention, a serum-free media may be used to culture intestinal epithelial cells. For example, media such as M199 and RPMI 1640 may be used which are specifically formulated for culturing mammalian cells in vitro without the addition of animal serum. Such media typically contain an extensive range of amino acids, vitamins, nucleic acid derivatives, growth factors and lipids (Morgan et al. 1950). As is shown in the following examples, in some embodiments M199 or RMPI 1640 alone, without the supplement of serum, will support the survival of SCBN cells with cell morphology similar to that of cells cultured in DMEM supplemented with serum but with a slower growth rate. Alternative serum-free media may be formulated in accordance with the use of alternative cell lines or culture conditions.

In some media for use in various aspects of the invention, supplements may be used to enhance or support cell viability. For example, glutamine may be added to a media for intestinal cells because it is generally regarded as a preferential nutrient for such cells (Wiren et al. 1998). In addition to glutamine, pyruvic acid and sucrose may also be added. As illustrated in the following examples, media supplemented with glutamine, sucrose, and pyruvic acid supports the growth of SCBN cells, wherein a significant proportion of the cells will form long microvilli. In some embodiments, the addition of glutamine, pyruvic acid, and sucrose into the culture medium may induce the formation of long microvilli, even where it does not appear to induce the expression of intestinal enzymes such as alkaline phosphatase and sucrase isomaltase.

In some embodiments, a fatty acid such as sodium butyrate may be added to cell media. Sodium butyrate is a short chain fatty acid that may be derived from bacterial fermentation of carbohydrates (Bond and Levitt, 1976). Sodium butyrate has been shown to induce in vitro cell differentiation in colorectal cancer cell lines such as CaCo2 and HT29. The effects of sodium butyrate on CaCo2 and HT29 cells include promotion of enzyme expression, tight junction formation, increase in TEER values, and apoptosis (Mariadason et al. 2000; Siavoshian et al. 2000).

In some embodiments, a kinase modulator such as Ro 31-8220 may be added to culture media. Ro 31-8220, has been reported to activate JNK1, in addition to inhibiting GRK-5, PKC, and MAPKAP kinase-1b and p70 S6 kinase (Beltman et al. 1996). In the following examples, the addition of 5 μM Ro 31-8220 alone tends to up-regulate the expression of alkaline phosphatase activity. However, in some embodiments, Ro 31-8220 alone may not induce sucrase isomaltase activity, nor increase the percentage of cells with long microvilli.

Microvilli SEM and TEER

To assay the structure of microvilli and to measure TEER, SCBN cells were cultured with DMEM media supplemented with FBS and collected at 24 hours post confluence. Harvested cells were seeded at the density of 1E5 cells/well in Falcon multiple insert plates (0.31 cm2/well). Five different conditions were tested here: 1) SCBN cells were seeded in non-coated insert wells with DMEM media supplemented with FBS and after 24 hours, media were changed with DMEM supplemented with FBS; 2) SCBN cells were seeded in rat-tail collagen-coated insert wells with DMEM media supplemented with FBS and after 24 hours, media were changed with base medium; 3) SCBN cells were seeded in rat-tail collagen-coated insert wells with DMEM media supplemented with FBS and after 24 hours, media were changed with base medium supplemented with 5 μM of Ro31-8220; 4) SCBN cells were seeded in rat-tail collagen-coated insert wells with DMEM media supplemented with FBS and after 24 hours, media were changed with base medium supplemented with 5 mM of Sodium butyrate, 5) SCBN cells were seeded in rat-tail collagen-coated insert wells with DMEM media supplemented with FBS and after 24 hours, media were changed with base medium supplemented with 5 μM of Ro31-8220 and 5 mM of sodium butyrate. Forty-eight hours after medium change, SCBN cells were processed for SEM examination following conventional SEM sample preparation. Assessments of microvilli structure were made from SEM images.

Microvilli were found to be short (<0.1 um, Table 1) when SCBN cells were cultured in DMEM media supplemented with 10% FBS. In base medium, about 30% of SCBN cells show long microvilli (>1 um, Table 1). The addition of Ro-31-8220 alone did not measurably increase the length of microvilli, nor the percentage of cells with long microvilli (Table 1). The addition of sodium butyrate to the base media reduces the length and numbers of microvilli on SCBN cells (<0.1 um, Table 1). However, SCBN cells cultured with base media supplemented with Ro-31-8220 and sodium butyrate show long microvilli (>1 um, Table 1).

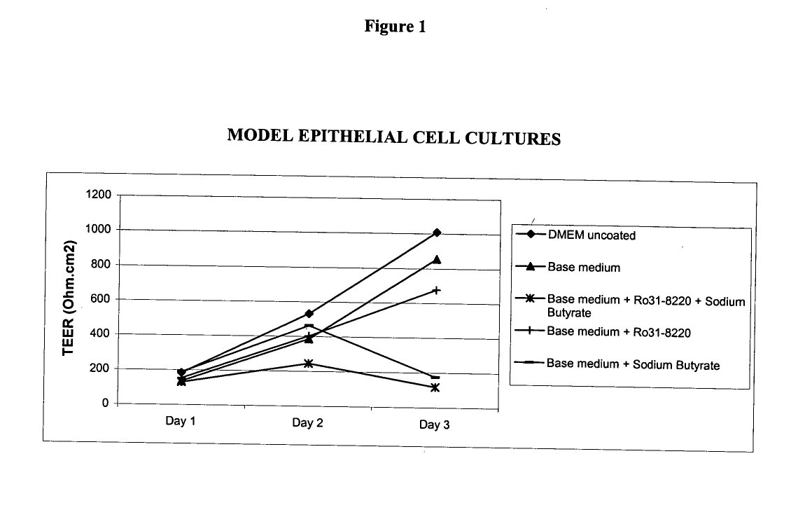

When SCBN cells were seeded in non-coated Falcon multi-insert wells with DMEM media supplemented with 10% FBS, the TEER values increased from 183 ohm.cm² at 24 hours after seeding to over 1000 ohm.cm² after 3 days (FIG. 1). When SCBN cells were seeded in rat-tail collagen-coated insert wells with DMEM media supplemented with FBS and then changed into base media, TEER values started at around 136 ohm.cm² at day 1 and then increased to about 850 ohm.cm² at day 3. When the base media were supplemented with 5 μM of Ro31-8220, the TEER value decreased to about 680 ohm.cm²; however, if the base media were supplemented with 5 mM sodium butyrate, TEER value decreased to about 175 ohm.cm² at day 3. When 5 μM of Ro31-8220 and 5 mM of sodium butyrate were added to the base medium, TEER value started at about 130 ohm.cm² and increased to about 250 ohm.cm² on day 2 and decreased to 119 ohm.cm² on day 3 (FIG. 1).

Enzyme Expression

To assay patterns of enzyme expression, SCBN cells were cultured with DMEM media supplemented with FBS and collected at 24 hours post confluence. SCBN cells were seeded with DMEM supplemented with FBS at 1E5 cells/well in non-coated and rat-tail collagen-coated Falcon multiple insert plate. Twenty-four hours after seeding media were change with DMEM supplemented with FBS with DMEM supplemented with FBS in non-coated inert wells whereas the media in rat-tail collagen-coated wells were changed with Kinetana base media supplemented with 5 μM of Ro31-8220 and 5 mM of sodium butyrate. Forty-eight hours after medium change, SCBN cells were lysed with ⅕ strength of Tris buffered saline on ice and the whole cell lysate was assayed for enzyme activity by incubating the cell lysate with specific substrate for 60 minutes at 37° C. Activity of Alkaline phosphatase (AP) was assayed with p-nitrophenylphosphate as substrate; dipeptidylpeptidase-IV (DPP) used glycyl-L-proline-4-nitroanilide as substrate; aminopeptidase N (APN) used L-alanine-p-nitroanilide as substrate; aminopeptidase A (APA) used y-glutamyl-p-nitroanilid as substrate. Total protein was assayed by using Bio-Rad protein assay kit.

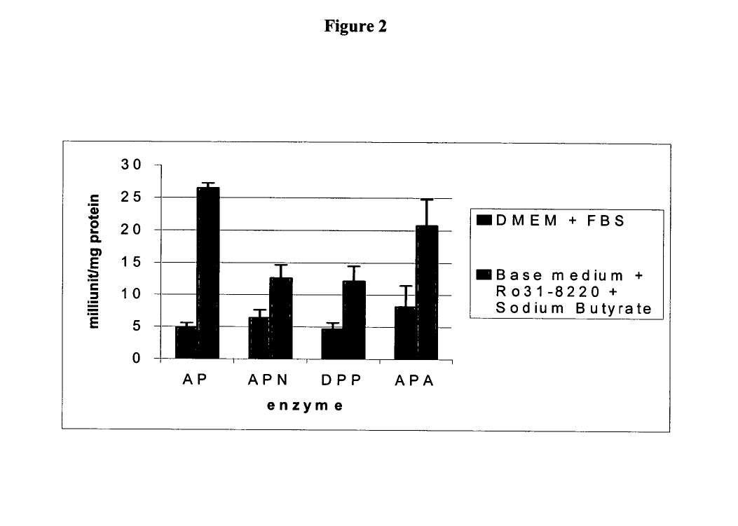

Activities of alkaline phosphatase (AP), dipeptidylpeptidase IV (DPP IV), aminopeptidase N (APN), and aminopeptidase A (APA) were present in SCBN cells and the enzymatic activities were increased when SCBN cells were cultured under culture conditions of the invention (FIG. 2).

Cell Differentiation under the Conditions of the Invention.

Advantages of Using the Invention over CaCo-2 Cells

Non-tumorigenic cells may be more representative of normal cells in vivo than tumorigenic cells such as CaCo-2. CaCo-2 cells have been used extensively in the pharmaceutical industry for absorption screening study. Differentiated CaCo-2 cells show characteristics of intestinal epithelial cells (Quaroni and Hochman 1996). However, recent microarray studies still show some discrepancies in the gene expression profile between differentiated CaCo-2 cells and human duodenum (Sun et al. 2002).

Tissue cultured cell lines have been used extensively in understanding the gene expression underlying the cell differentiation. Most of these cell lines are derived from tumors, such as CaCo-2 cells which are derived from colorectal tumors (Quaroni and Hochman 1996). Those studies using tumor cells provide a lot of clues into cell differentiation. However, sometimes the results can also be contradictory due to the nature of cell lines in use. PI3K has been implicated in the involvement of cell differentiation of intestinal epithelial cells, however, inhibition of PI3k induced cell differentiation in CaCo-2 cells is reported by one group of researchers (Wang et al. 2001, 2002, Kim et al. 2002) while activation of PI3K induced cell differentiation in CaCo-2 is reported by another group (Laperise et al 2002).

Several gene products are reported to be involved in differentiation of intestinal epithelial cells. Cdx1 and cdx2 are two homeobox genes expressed in the intestinal epithelial cells. During the differentiation process, the expression of cdx 2gene is up-regulated and that of cdx1 is down-regulated. The up-regulation of cdx2 induces the expression of intestinal digestive enzyme, like sucrase-isomaltase and other differentiation characteristics in intestinal epithelial cells, like long microvilli (Freund et al. 1998).

Catenin is another gene product reported to be involved in cell differentiation (.Mariadason et al 2001). Cell-cell contact increases the formation adherens junctions. The formation of adherens junctions recruits beta-catenin to the cell membrane, which leads to the decrease in the beta-catenin/TCF-4 complex in the nucleus (Mariadason et al 2001). The increase in the formation of adherens junctions on the cell membrane activates the p38 MAPK cascade, which could enhance the expression of cdx2 (Laprise et al 2002). The target of catenin/TCF-4 complex is c-MYC. The decrease in catenin/TCF-4 activity leads to the decrease in the expression of c-MYC which releases p21^{CIP/WAF1}. p2^{CIP/WAF1} mediates G1 arrest and differentiation (van de Wetering et al 2002).

The decrease in cytosol free beta-catenin can come from another direction. During differentiation, the increase in the expression of phosphatase and tensin homologue deleted on chromsome 10 (PTEN) inhibits phatidylinositol (PI) 3-kinase (PI3K)(Kim et al 2002), which leads to the activation of glycogen synthase kinase-3 (GSK-3)(Wang et al 2001, 2002). GSK-3 forms complex with APC proteins, axin/conductin, and beta-catenin. Catenin in this complex is phosphorylated by GSK3 and targeted for destruction by proteasome. Thus free beta-catenin concentration in cytosol is further decreased, which leads to cell cycle arrests (Frame and Cohen 2001).

Since c-MYC is also the down stream target for ERK/MEK pathway which is the down stream of ras (Sears et al 2000) and ras has been implicated in the regulation of PI3K activity (Soubeyran et al 2001). ras is the up stream of MAPK and PKC and all of these kinases have been implicated in the regulation of cell differentiation (Lorentz et al 1999). Thus, a cross talk among ERK/MEK, PI3K, MAPK, and PKC is possible and warrant for further study.

SCBN cells are derived from normal tissue and are non-tumorigenic when injected into nude mice. They can be manipulated into differentiation with longer microvilli. Thus, this cell line provides a unique chance for looking into gene expression during cell differentiation of human intestinal epithelial cells.

Microarray will be used to examine the gene expression pattern during the cell differentiation. The result from microarray study will be further verified by northern blotting, cloning and sequencing to establish the signaling pathways involved in the differentiation of intestinal epithelial cells.

Methods:

Preparation of Total mRNA

SimBioDAS® cells are cultured according to the procedures mentioned in the patent application. Cells are harvested by trypsinization and then seeded into collagen-coated Petri-dishes with regular serum containing growth media. Twenty-four hours after seeding, media are changed with differentiation media with RO 31-8220 and sodium butyrate. Total mRNA are extracted from cells 24 hours after seeding into the collagen-coated Petri dishes and 24 hours after changing into differentiation media (48 hours after seeding).

TriZol reagent will be used to extract the total mRNA and follow the maufacturer's protocol (Gibco BRL). Total mRNA is further cleaned with RNeasy mini kit (Qiagen). cDNA synthesis will use SuperScript Choice system Gibco BRL (Grand Island, N.Y., USA). BioArray high yield RNA transcription labeling kit from Enzo Biochem will be used to synthesize the biotin-labeled cRNA for Genechip study.

Microarray

A commercially available human gene chip will be used for the microarray study, such as Affymetrix human GeneChip set. Microarray study will follow standard manufacturer's protocols.

Cloning and Sequencing

Further validation of the gene products by northern blotting, cloning, and sequencing of gene products will follow the Sambrook et al (2001).

Functional Validation of the Presence of Specific Proteins

The function of proteins (transporters) will be assayed by the comparison of the directional transport of their specific substrates in the presence and absence of inhibitors, i.e. A-B/B-A studies with and without inhibitors.

The presence of metabolic enzymes will be assayed by the incubations with the cells of the invention with specific enzyme substrates and then assay for the possible metabolites through LC/MASS or other related analytical methods.

For the junctional proteins, we will use the anti-sense RNA techniques or transfect the dominant negative genes to silence the expression of particular genes (Sambrook et al 2001) and then assay their contribution to junctional formation and passive absorption through our standard permeability study.

Expected Results

Genes expressed in the human duodenum epithelial cells have been identified by gene microarray study (Sun et al. 2002). These genes could possibly be expressed by the cells of the invention since these cells are derived from human duodenum. Possible genes expressed by cells of the invention that are important for defining the permeability properties of cell monolayers of the invention can be classified into three categories (Table 2).

First is the transporter category, which is separated into two big families: ABC super family and Solute-carrier family. Functions of these are contributions to active processes of drug absorption. The relative amount of transporter expression in this the SimBioDAS® cells after being subjected to the differentiation medium will be unique to the in vitro monolayer and defines the active permeability process for the cell monolayer.

Second is the metabolic enzyme category, which contributes to the first pass metabolism of drug absorption. The relative amount of metabolic enzyme expression in this the SimBioDAS® cells after being subjected to the differentiation medium will be unique to the in vitro monolayer and defines the first pass metabolism process for the monolayer.

Third is the junctional protein category, which defines the junctional formation and properties. Their relative level of gene expression contributes to the tightness of junctions and hence passive processes of drug absorption (Yu 2000). Thus, the relative amount of tight junction protein expression in this the SimBioDAS® cells after being subjected to the differentiation medium will be unique to the in vitro monolayer and defines the paracellular permeability process for the monolayer.

The comparison in the gene expression profiles between differentiation medium treated and untreated cells will provide information on the cell signaling pathways involved in the SimBioDAS® cell differentiation.

After the identification of the signaling pathway involved in the SimBioDAS® cell differentiation, the knowledge on the signaling pathways in SimBioDAS® cells can be use to manipulate the cell differentiation in SimBioDAS® cells. The manipulation can either be genetic manipulation or the addition of different combinations of kinase activators and kinase inhibitors into the culture medium. The perturbation of cell signaling pathways involved in cell differentiation can lead to more fully differentiated SimBioDAS® cells, i.e. higher percentage of cells expressing long microvilli, brush border enzymes, and transporters.

The genetic manipulation could include ectopic transfection of certain genes identified by the above microarray study, which could interfere with the cell signaling pathways and hence lead to further cell differentiation. The genetic manipulation will follow Sambrook et al (2001).

Perturbation of the signaling pathways can also be adding kinase inhibitors alone or in combination of kinase activators into the tissue culture media. The kinase inhibitors may include chemicals that inhibit signaling pathways leading to cell proliferation and apoptosis at different signaling cascade level. The kinase activators may include chemicals that activate signaling pathways leading to cell-cycle arrest and differentiation at different signaling pathway cascade level.

Characteristics of the Invention

1. Distinctive Paracellular Diffusion Performance of Kinetana Cell Line under Differentiation Conditions of the Invention

The proprietary growth conditions result in a different paracellular diffusion performance of the Kinetana cell line compared with CaCo-2 and T-84 human colon cancer cell lines (He, Murby et al. 1998) and also SCBN with the Buret condition. The proprietary condition correlates better with human paracellular marker permeability, and this appears to relate to the differences in tight junction pore size between the new method and the others. The evidence for these claims is as follows.

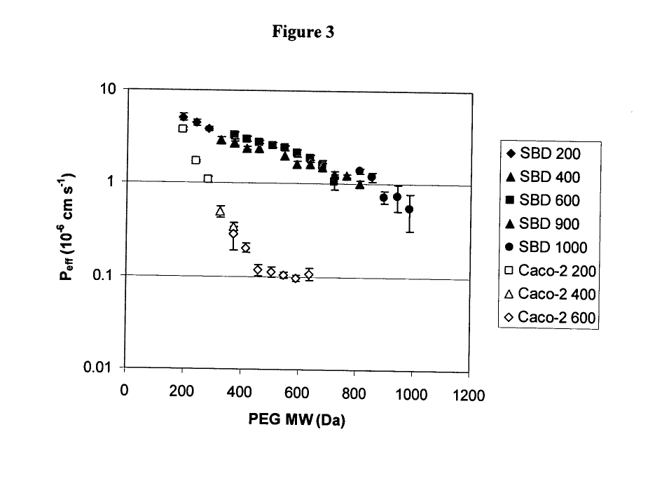

(He, Murby et al. 1998) relate the permeability profile of PEG oligomers to tight junction pore size. Their data in CaCo-2 correspond very closely to Kinetana's data with Caco-2, in that there is a steep drop-off in permeability between molecular weights 200 and 450, then a leveling off. The invention permeability, in contrast, declines slowly and continuously from MW 200 to over 1,000 Da (FIG. 3). These data imply that the higher leakiness of the invention membranes arises from larger tight junction pore size and hence molecular selectivity.

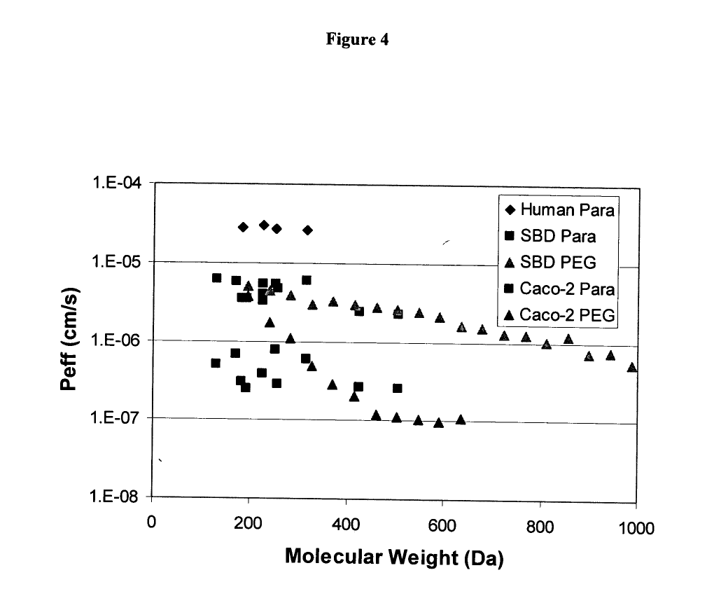

With the invention culture conditions, paracellular drug substrate permeabilities correlate well with PEG data, while in Caco-2, there is more scatter and the data correlate poorly to the PEG data (FIG. 4). The sparse human in vivo data visually show a gradual decline in paracellular substrate permeability with increasing molecular weight, with a similar slope to that observed with the invention, but not with Caco-2 (Sun, Lennernas et al. 2002). This is additional evidence that the invention performs more consistently and models human paracellular diffusion in a more physiological manner than does Caco-2.

Under the conditions of the invention, the permeability of PEG 900 is close to 1E-6 cm/sec, which predicts the human bioavailability of this oligomer to be around 12%. The bioavailability of PEG 900 is reported to be 10% (Yee 1997). In contrast, the permeability of PEG 900 was too low to be measured in Caco-2 (FIGS. 3, 4) and also in Kinetana cells using the Buret growth condition.

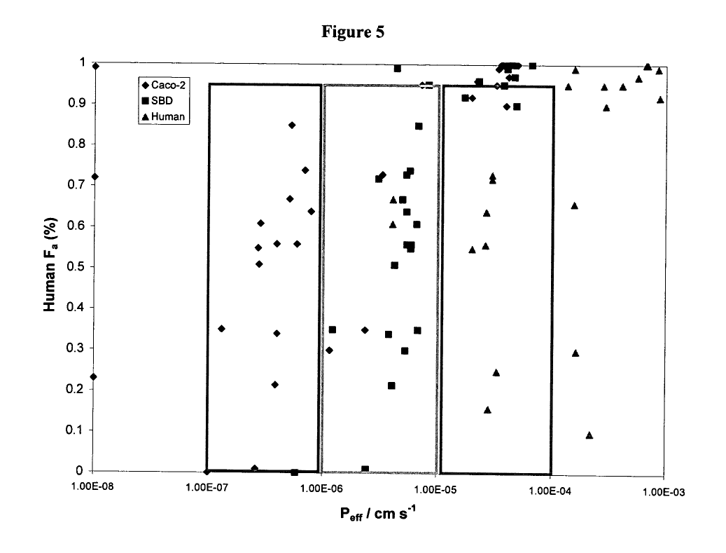

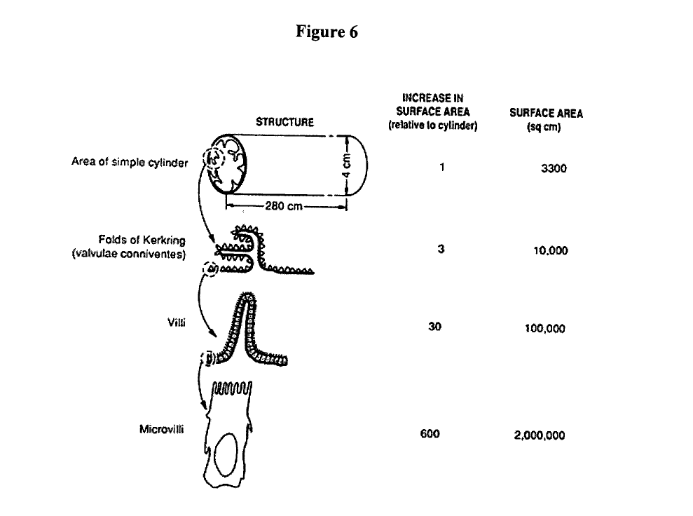

The invention cells grown under the proprietary condition have a narrow range of paracellular permeability (1E-6-1E-5 cm/sec, see middle box in FIG. 5) with no outliers. The permeability of these cells correlates directly to the human permeability range (Sun, Lennernas et al. 2002) with only an area correction that accounts for the difference between a flat monolayer and the surface area of an equivalent area of intestine, which contains villi and folds that increase the surface area 10-30-fold (FIG. 6). In contrast, Caco-2 permeabilities for these substrates have a wide range that exceeds the 1E-7-1E-6 range (see left box, FIG. 5) and require a larger than anatomical correction factor to correlate to human data (70-130-fold, see right box, FIG. 5).

1. Distinctive Efflux Transporter Performance of Kinetana Cell Line under Differentiation Conditions of the Invention

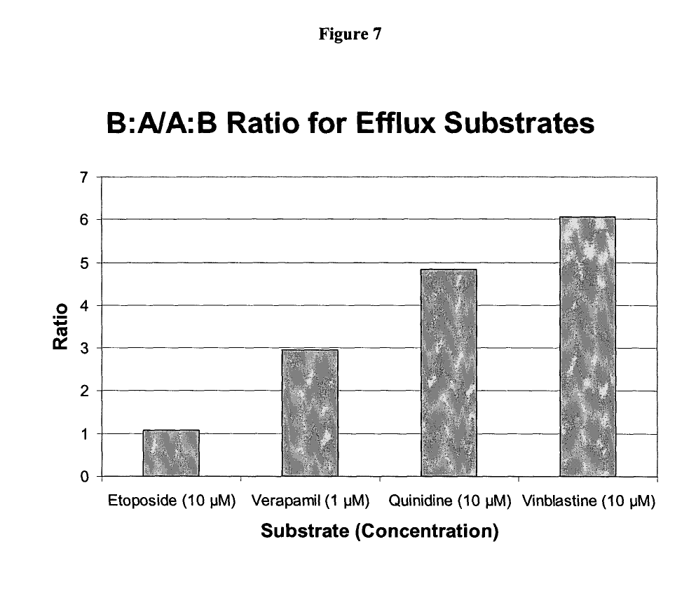

Cells of the invention show functional evidence of efflux transporter activity when cultured under the proprietary conditions. To establish a preliminary characterization of efflux transporter activity, Kinetana measured the B:A/A:B ratios of four efflux substrates, etoposide, verapamil, quinidine and vinblastine. The results are shown in FIG. 7. Of the four substrates, only etoposide had a ratio of less than 2, and it was measured at a donor side concentration of 10 μM (perhaps above the Kₘ of the transporter for this substrate). A ratio greater than 2 (some suggest a ratio as low as 1.3) is normally considered to suggest efflux pump activity in Caco-2, but the ratio will be inherently lower in the invention because paracellular diffusion is accounted for due to its leakier tight junctions. Conversely, any given B:A/A:B ratio in the invention will reflect a higher level of expression of efflux transporter activity. Levels of transporter activity of the invention are in general about 50% of those of Caco-2. It is not clear to what extent the expression of transporter is appropriate to reflect the in vivo condition; however, it should be noted that Caco-2 is widely considered to over-express the efflux transporter P-glycoprotein.

Another issue related to claims is the identity of the efflux transporter(s) expressed in the invention or at least the pattern of transporter gene expression. The B:A/A:B ratios are not uniformly less than those observed in Caco-2 for the different substrates. Therefore, the transporters may be different. Additional evidence that efflux transporter gene expression patterns may differ from those of other cell lines is provided by Western blotting of MDR by two different monoclonal antibodies (JSB-1 from Research Diagnostics, Inc. and QCRL-1 from ATCC). Whereas these antibodies show bands at 170 and 190 kDa, respectively with Caco-2, with the invention system, they showed a single band at 110 kDa, supporting the expression of an isoform of MDR in the Kinetana cell line.

Applications of the Invention

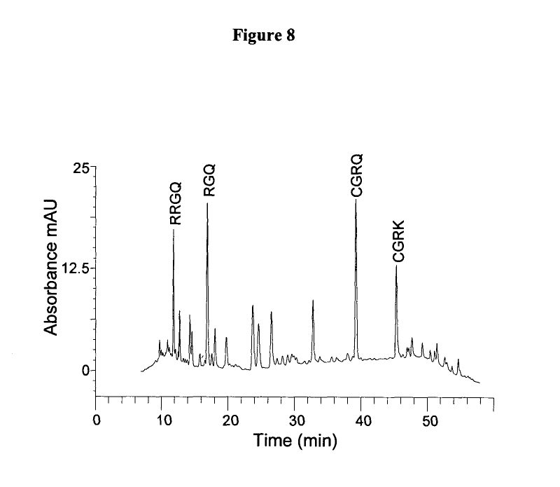

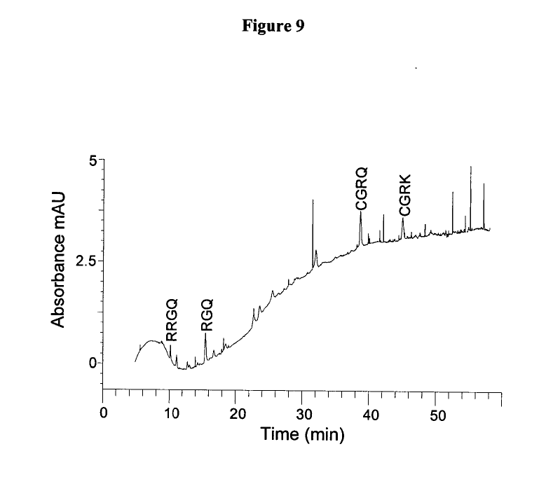

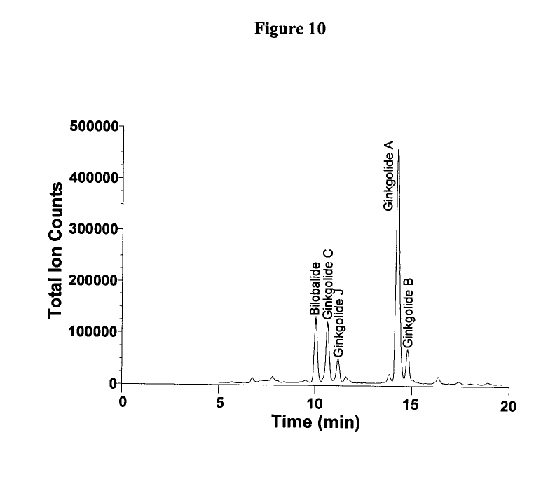

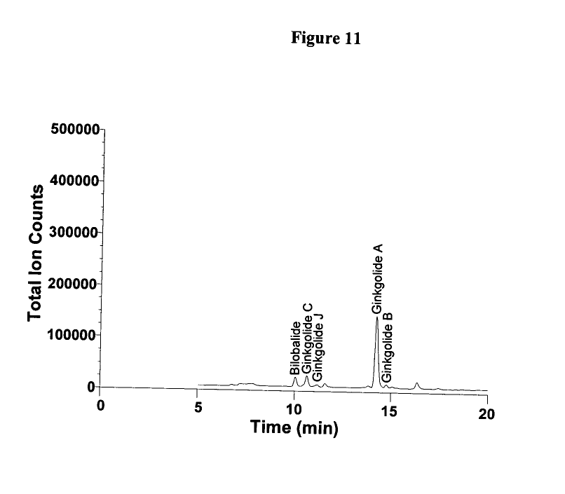

Although the invention may be used to conduct permeability screening in a manner similar to how Caco-2 the industry standard is used, it has a unique and novel application in identifying absorbable active ingredients in natural health products (or natural products or nutraceuticals or botanicals or herbals or dietary supplements) or any products that contain mixtures of ingredients of interest (as marker substances or active ingredients) or botanicals. The idea is as follows. Similar to pharmaceutical development, components are not considered active unless they are absorbed into the blood stream. A number of methods have been developed to measure the activities of botanical extracts; very few have measured the active components, let alone absorbable components that are active. It has been shown time and again that marker components that have in vitro activities are not absorbed. The active ingredient profile determined using in vitro methods is usually erroneous; this is particularly true for the proportion of the ingredients required because the absorption of individual components is potentially different. A good example is the determination of the ingredient profiles of Ginkgo biloba. People think that the ratios of the ingredients in the herb are important (See FIGS. 8, 10, Tables 3, 4, apical side ratios). But it is only the absorbed ingredients that can have activity and not all ingredients are absorbed or absorbed at the same rate. Therefore, the profiles of absorbed ingredients will differ from the profiles of ingredients in the product, and it is the absorbed ingredient profiles that are biologically most relevant. The invention can be used to generate in vitro “absorbed ingredient” profiles of ingredients that pass from the apical to the basal side of the invention, which is analogous to what occurs in vivo. Tables 3 and 4 show how the profiles of ingredients differ between the apical side of the invention (pre-absorption, i.e. the product ingredients) and the basal side (absorbed ingredients) for two classes of ingredients in a Ginkgo product. The basal side chromatograms in FIGS. 9 and 11 depict the post-absorption analytical profiles of the ingredients profiled in FIGS. 8 and 10. These profiles are characteristic to the invention, and also to the product that contains well defined ratios of absorbable active ingredients. It should be pointed out that the ratios of these ingredients are not constant; it is dependent on the growing conditions, etc., for instance the plant from which the extract is derived. In order to maintain fixed ratios, the herb may have to be blended with crops from different harvests. Using this method to determine the biological activity of mixture products provides a useful means to characterize and monitor their quality. In addition, with an optimized fixed ratio of absorbed ingredients, the pre-absorption ratios of ingredients of a product with a particular formulation may be back-calculated. By this method, Kinetana has developed a proprietary formulation of Ginkgo biloba with a fixed ingredient profile. The percentages of the various terpene lactones in the formulation are as follows:

| | |
|---|---|
| Bilobalide | 1.2 +/− 0.48% |
| Ginkgolide A | 2 +/− 0.8% |
| Ginkgolide B | 1 +/− 0.4% |
| Ginkgolide C | 0.2 +/− 0.08% |
| Ginkgolide J | 0.6 +/− 0.24% |

TABLE 1
Summary of Culture Conditions in Various
Embodiments of the Present Invention

| | EXTRACELLULAR | | |
|---|---|---|---|
| MEDIA | MATRIX COATING | COMPRISES | OBSERVATIONS |
| DMEM + FBS | None | DMEM | Medium used in |
| | | 10% fetal | previously |
| | | bovine serum | published |
| | | L-glutamine (2 mM) | studies with |
| | | MEM Non- | SCBN cells. |
| | | essential amino | SCBN cells |
| | | acids (1x) | show short and |
| | | HEPES 10 mM | spare |
| | | | microvilli with |
| | | | high TEER value |
| | | | and low |
| | | | enzymatic |
| | | | activity |
| base medium | Rat-tail | M199, DMEM, | SCBN cells show |
| | collagen | RPMI1640 (v:v:v = 4:3:3) | long microvilli |
| | | D-glucose (4.5 g/L) | but with high |
| | | L-glutamine (4 mM) | TEER value |
| | | sucrose (5 mM) | |
| | | pyruvic acid | |
| | | (4 mM) | |
| | | ITS 1X | |
| | | 1% Non- | |
| | | essential amino | |
| | | acids | |
| base medium + Ro31-8220 | Rat-tail | base medium + 5 μM | SCBN cells show |
| | collagen | Ro31-8220 | long microvilli |
| | | | but with high |
| | | | TEER value |
| base medium + Sodium | Rat-tail | base medium + 5 mM | SCBN cells show |
| Butyrate | collagen | Sodium | short/no |
| | | Butyrate | microvilli but |
| | | | with low TEER |
| | | | value |
| base medium + Ro31-8220 + Sodium | Rat-tail | base medium + 5 μM | SCBN cells show |
| Butyrate | collagen | Ro31-8220 + 5 mM | long |
| | | Sodium | microvilli, low |
| | | Butyrate | TEER value, and |
| | | | increased |
| | | | enzymatic |
| | | | activity |

TABLE 2
List of the most important genes expressed in the
invention, by category

Transporters:
a) ATP-binding cassette super-family
ABCB1 = ATP-binding cassette, sub-family B
(MDR/TAP), member 1 (MDR1)
ABCB4 = ATP-binding cassette, sub-family B
(MDR/TAP), member 4 (MDR2/3)
ABCB10 = ATP-binding cassette, sub-family B
(MDR/TAP), member 10
ABCB11 = ATP-binding cassette, sub-family B
(MDR/TAP), member 11 (BSEP)
ABCC3 = ATP-binding cassette, sub-family C
(CFTR/MRP), member 3 (cMOAT2/MRP3)
ABCC4 = ATP-binding cassette, sub-family C
(CFTR/MRP), member 4 (MRP4)
ABCC5 = ATP-binding cassette, sub-family C
(CFTR/MRP), member 5 (MOAT-c/MRP5)
ABCC6 = ATP-binding cassette, sub-family C
(CFTR/MRP), member 6 (MRP6)
ABCC11 = ATP-binding cassette, sub-family C
(CFTR/MRP), member 11 (MRP8)
ABCG2 = ATP-binding cassette, sub-family G (WHITE),
member 2
ABCG5 = ATP-binding cassette, sub-family G (WHITE),
member 5 (sterolin 1)
ABCG8 = ATP-binding cassette, sub-family G (WHITE),
member 8 (sterolin 2)
b) Solute-carrier family
SLC2A2 = solute carrier family 2 (facilitated
glucose transporter), member 2
SLC2A5 = solute carrier family 2 (facilitated
glucose/fructose transporter), member 5
SLC2A8 = solute carrier family 2, (facilitated
glucose transporter) member 8
SLC3A1 = solute carrier family 3 (cystine, dibasic
and neutral amino acid transporters,
activator of cystine, dibasic and neutral amino acid
transport), member 1
SLC5A1 = solute carrier family 5 (sodium/glucose
cotransporter), member 1
SLC5A4 = solute carrier family 5 (low affinity
glucose cotransporter), member 4
SLC5A6 = solute carrier family 5 (sodium-dependent
vitamin transporter), member 6
SLC7A1 = solute carrier family 7 (cationic amino
acid transporter, y+ system), member 1
SLC7A2 = solute carrier family 7 (cationic amino
acid transporter, y+ system), member 2
SLC7A3 = solute carrier family 7 (cationic amino
acid transporter, y+ system), member 3
SLC7A4 = solute carrier family 7 (cationic amino
acid transporter, y+ system), member 4
SLC7A5 = solute carrier family 7 (cationic amino
acid transporter, y+ system), member 5
SLC7A7 = solute carrier family 7 (cationic amino
acid transporter, y+ system), member 7
SLC7A8 = solute carrier family 7 (cationic amino
acid transporter, y+ system), member 8
SLC7A9 = solute carrier family 7 (cationic amino
acid transporter, y+ system), member 9
SLC7A10 = solute carrier family 7, (cationic amino
acid transporter, y+ system) member 10
SLC7A11 = solute carrier family 7, (cationic amino
acid transporter, y+ system) member 11
SLC9A2 = solute carrier family 9 (sodium/hydrogen
exchanger), isoform 2
SLC10A2 = solute carrier family 10 (sodium/bile acid
cotransporter family), member 2
SLC15A1 = solute carrier family 15 (oligopeptide
transporter), member 1 (PEPT1)
SLC15A2 = solute carrier family 15 (H+/peptide
transporter), member 2 (PEPT2)
SLC16A4 = solute carrier family 16 (monocarboxylic
acid transporters), member 4
SLC16A5 = solute carrier family 16 (monocarboxylic
acid transporters), member 5 (MCT5/6)
SLC16A7 = solute carrier family 16 (monocarboxylic
acid transporters), member 7 (MCT2)
SLC16A8 = solute carrier 16 (monocarboxylic acid
transporters), member 8 (MCT3)
SLC21A2 = solute carrier family 21 (prostaglandin
transporter), member 2
SLC21A6 = solute carrier family 21 (organic anion
transporter), member 6
SLC23A2 = solute carrier family 23 (nucleobase
transporters), member 2
SLC34A2 = solute carrier family 34 (sodium
phosphate), member 2
SLC35A2 = solute carrier family 35 (UDP-galactose
transporter), member A2
CETP = cholesteryl ester transfer protein
COMT = catechol-O-methyltransferase
Metabolic enzymes
CYP2A13 = cytochrome P450, family 2, subfamily A,
polypeptide 13
CYP3A4 = cytochrome P450, family 3, subfamily A,
polypeptide 4
CYP3A7 = cytochrome P450, family 3, subfamily A,
polypeptide 7
CYP4F12 = cytochrome P450, family 4, subfamily F,
polypeptide 12
CYP7B1 = cytochrome P450, family 7, subfamily B,
polypeptide 1
FMO1 = Flavin-containing mono-oxygenase 1
FMO2 = Flavin-containing mono-oxygenase 2
FMO3 = Flavin-containing mono-oxygenase 3
FMO4 = Flavin-containing mono-oxygenase 4
FMO5 = Flavin-containing mono-oxygenase 5
MAOA = monoamine oxidase A
MAOB = monoamine oxidase B
UGT1A1 = UDP glycosyltransferase 1 family,
polypeptide A1
UGT1A2 = UDP glycosyltransferase 1 family,
polypeptide A2
UGT1A3 = UDP glycosyltransferase 1 family,
polypeptide A3
UGT1A4 = UDP glycosyltransferase 1 family,
polypeptide A4
UGT1A5 = UDP glycosyltransferase 1 family,
polypeptide A5
UGT1A6 = UDP glycosyltransferase 1 family,
polypeptide A6
UGT2B15 = UDP glycosyltransferase 2 family,
polypeptide B15
GSTA1 = glutathione S-transferase A1
GSTA2 = glutathione S-transferase A2
APOA1 = apolipoprotein A-I
APOBEC1 = apolipoprotein B mRNA editing enzyme,
catalytic polypeptide 1
APOL2 = apolipoprotein L, 2
Junctional protein genes
TJP1 = tight junction protein 1 (zona occludens 1)
TJP2 = tight junction protein 2 (zona occludens 2)
TJP3 = tight junction protein 3 (zona occludens 3)
TJP4 = tight junction protein 4 (peripheral)
OCLN = occludin
CLDN 1-20 = claudin 1-20
CDH1 = cadherin 1, type 1, E-cadherin (epithelial)
CD36 = CD36 antigen (collagen type I receptor,
thrombospondin receptor)
ITGB1 = integrin, beta 1 (fibronectin receptor, beta
polypeptide, antigen CD29 includes
MDF2, MSK12)
ITGB6 = integrin, beta 6
DSC1 = desmocollin 1
DSG3 = desmoglein 3 (pemphigus vulgaris antigen)

TABLE 3
Profile of Ginkgo terpene lactone components ratio
to Ginkgolide A*

| | |
|---|---|
| Apical side | Basal side |

| | Mean | STDEV | Mean | STDEV |
|---|---|---|---|---|
| Bilobalide | 0.66 | 0.02 | 0.44 | 0.02 |
| Ginkgolide C | 0.32 | 0.01 | 0.20 | 0.01 |
| Ginkgolide J | 0.86 | 0.02 | 0.55 | 0.02 |
| Ginkgolide A | 1.00 | 0.00 | 1.00 | 0.00 |
| Ginkgolide B | 0.20 | 0.01 | 0.12 | 0.01 |

*Data were collected from six commercial ginkgo extract samples

TABLE 4
Profile of Ginkgo intact flavone components ratio
to Rutin*

| | |
|---|---|
| Apical | basal |

| | Mean | STDEV | Mean | STDEV |
|---|---|---|---|---|
| Rutin | 1.00 | 0.000 | 1.00 | 0.00 |
| CGRQ | 0.17 | 0.001 | 1.19 | 0.35 |
| CGRK | 0.13 | 0.002 | 1.00 | 0.20 |

*Data were collected from six commercial ginkgo extract samples Amount of RRGQ in the basal side is too small to be quantified

References

The following documents are incorporated herein by reference:

Beltman, J.McCormick, F., and Cook, S. J. (1996) The selective protein kinase C inhibitor, Ro-31-8220, inhibits mitogen-activated protein kinase phosphatase-1 (MKP-1) expression, induces c-Jun expression, and activates Jun N-terminal kinase. J Biol Chem 271 (43): 27018-24.

Eckert R, Randall D. (1983) Animal physiology: mechanisms and adaptations. 2nd ed, Freeman and company, New York.

Engman, H. A., et al., CYP3A4, CYP3A5, and MDR1 in human small and large intestinal cell lines suitable for drug transport studies. J Pharm Sci, 2001. 90(11): p. 1736-51.

Frame and Cohen 2001. GSK3 takes centre stage more than 20 years after its discovery. Biochem. J. 359: 1-16.

Freund et al. 1998. The cdx-1 and cdx-2 homeobox genes in the intestine. Biochem. Cell Biol. 76: 957-969.

He, Y. L., S. Murby, et al. (1998). “Species differences in size discrimination in the paracellular pathway reflected by oral bioavailability of poly(ethylene glycol) and D-peptides.” J Pharm Sci 87(5): 626-33.

Hilgendorf, C., et al., Caco-2 versus Caco-2/HT29-MTX co-cultured cell lines: permeabilities via diffusion, inside- and outside-directed carrier-mediated transport. J Pharm Sci, 2000. 89(1): p. 63-75.

Kim et al. 2002. PTEN and TNF-alpha regulation of the intestinal-specific cdx-2 homeobox gene through a PI3K, PKB/Akt, and NF-kB-dependent pathway. Gastroenterology 123: 1163-1178.

Laprise et al. 2002. Phosphatidylinositol 3-kinase controls human intestinal epithelial cell differentiation by promoting adherens junction assembly and p38 MAPK activation. J. Biol. Chem. 277(10): 8226-8234.

Lorentz et al 1999. Downregulation of the colon tumour-suppressor homeobox gene Cdx-2 by oncogenic ras. Oncogene 18: 87-92.

Madara, J. L. and Trier, J. S. (1987) Functional morphology of the mucosa of the small intestine. In Physiology of the gastrointestinal tract, 2nd edition, ed. Johnson, L. R., Raven Press, New York

Mariadason et al. 2000. Divergent phenotypic patterns and commitment to apoptosis of Caco-2 cells during spontaneous and butyrate-induced differentiation. J Cell Physiol. 183: 347-54

Mariadason et al. 2001. Down-regulation of beta-catenin TCF signaling is linked to colonic epithelial cell differentiation. Cancer Res. 61: 3465-3471.

Moore, J. B. et al., Inhibition of PTH desensitization by inhibition of the G protein-coupled receptor kinase-5 enzyme with Ro 32-0432, FASEB J. Part II 12, A741 (1998).

Morgan, J. F., H. J. Morton, and R. C. Parker, Nutrition of animal cells in tissue culture. I. Initial studies on a synthetic medium. Proceedings of the Society for Experimental Biology and Medicine, 1950. 73(1): p. 1-8.

O'Loughlin, E.V., et al., Interleukin 2 modulates ion secretion and cell proliferation in cultured human small intestinal enterocytes. Gut, 2001. 49(5): p. 636-43.

Owens R B, Smith H S, Nelson-Rees W A, Springer E L.(1976) Epithelial cell cultures from normal and cancerous human tissues. J Natl Cancer Inst 56(4):843-9

Pang, G., et al., Immunologic, functional, and morphological characterization of three new human small intestinal epithelial cell lines. Gastroenterology, 1996. 111(1): p. 8-18.

Pontier C, Pachot J, Botham R, Lenfant B, Arnaud P. (2001) HT29-MTX and CaCo-2/TC7 monolayers as predictive models for human intestinal absorption: role of the mucus layer. J Pharm Sci 90(10):1608-19

Quaroni A, Hochman J. (1996) Development of intestinal cell culture models for drug transport and metabolism studies. Adv Drug Del Rev 22:3-52.

Ringer J. A.(2001) Utility of a Bioassay-Based Quality Standards Testing Program (BioFit) for Botanical Products in Examining The Science Behind Nutraceuticals. Proceedings of the AAPS Dietary Supplements Forum. P83-95.

Sambrook, J., Russell, D. W., and Sambrook, J. (2001) Molecular cloning: a laboratory manual. 3ed, Cold Spring Harbor Press.

Sears et al. 2000. Multiple Ras-dependent phsophorylation pathways regulate Myc protein stability. Genes Dev. 14: 2501-2514.

Siavoshian et al 2000. Butyrate and trichostatin A effects on the proliferation/differentiation of human intestinal epithelial cells: induction of cyclin D3 and p21 expression. Gut 46(4): p. 507-14)

Soubeyran et al 2001. Homeobox gene cdx1 regulates ras, rho, and PI3 kinase pathways leading to transformation and tumorigenesis of intestinal epithelial cells. Oncogene 20: 4180-4187.

Sun, D., H. Lennernas, et al. (2002). “Comparison of human duodenum and Caco-2 gene expression profiles for 12,000 gene sequences tags and correlation with permeability of 26 drugs.” Pharm Res 19(10): 1400-16.

Teoh D A, Kamieniecki D, Pang G, Buret A G. (2000) Giardia lamblia rearranges F-actin and alpha-actinin in human colonic and duodenal monolayers and reduces transepithelial electrical resistance. J Parasitol 86(4):800-6

Van de Wetering et al. 2002. The beta-catenin/TCF-4 complex imposes a crypt progenitor phenotype on colorectal cancer cells. Cell 111: 241-250.

Wang, et al. 2001. Inhibition of the phosphatidylinositol 3-kinase pathway contributes to HT29 and Caco-2 intestinal cell differentiation. Gastroenterology 120: 1381-1392.

Wang, et al. 2002. Regulation of TRAIL expression by the phosphatidylinositol 3-kinase/Akt/GSK-3 pathway in human colon cancer cells. J. Bio. Chem. 277(39): 36602-36610.

Wiren, M., K. E. Magnusson, and J. Larsson, The role of glutamine, serum and energy factors in growth of enterocyte-like cell lines. Int J Biochem Cell Biol, 1998. 30(12): p. 1331-6.

Yee, S. (1997). “In vitro permeability across Caco-2 cells (colonic) can predict in vivo (small intestinal) absorption in man—fact or myth.” Pharm Res 14(6): 763-6.

Yu, A S (2000) Paracellular solute transport: more than just a leak? Curr. Opin. Nephrol. Hypertens. 9(5): 513-515.

Conclusion

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

What is claimed is:

1. A method of culturing cells, comprising growing intestinal epithelial cells to form a confluent cell layer in a culture media, wherein the intestinal epithelial cells express an enzyme activity selected from the group consisting of alkaline phosphatase, dipeptidyl peptidase IV, aminopeptidase and amino peptidase A, and wherein the culture media comprises:
a) an amount of a differentiating compound effective to maintain a reduced or physiologically meaningful transepithelial electrical resistance of the confluent cell layer; and
b) an amount of a kinase modulator effective in combination with the differentiating compound to support growth of microvilli longer than 1 micrometer on a portion of the epithelial cells.

2. The method of claim 1, wherein the differentiating compound is a fatty acid.

3. The method of claim 1, wherein the differentiating compound is a butyrate salt.

4. The method of claim 1, wherein the differentiating compound is sodium butyrate.

5. The method of any one of claims 1 through 4, wherein the effective amount of the differentiating compound is greater than 1 mM.

6. The method of any one of claims 1 through 4, wherein the effective amount of the differentiating compound is from about 1 mM to about 10 mM.

7. The method of any one of claims 1 through 4, wherein the effective amount of the differentiating compound is about 5 mM.

8. The method of any one of claims 1 through 7, wherein the kinase modulator is a kinase inhibitor.

9. The method of any one of claims 1 through 7, wherein the kinase modulator has an activity selected from the group consisting of activating JNK1, inhibiting GRK-5, inhibiting PKC, inhibiting MAPKAP kinase-1beta and inhibiting p70 S6 kinase.

10. The method of any one of claims 1 through 7, wherein the kinase modulator is 2-{1-[3-(amidinothio)propyl]-1H-indol-3-yl}-3-(1-meethylindol-3-yl)-maleimide methanesulfonate.

11. The method of any one of claims 1 through 10, wherein the effective amount of the kinase modulator is greater than 1 micromolar.

12. The method of any one of claims 1 through 10, wherein the effective amount of the kinase modulator is from about 1 micromolar to about 10 micromolar.

13. The method of any one of claims 1 through 10, wherein the effective amount of the kinase modulator is about 5 micromolar.

14. The method of any one of claims 1 through 13, wherein the confluent cell layer is grown on an extracellular matrix base.

15. The method of claim 14, wherein the extracellular matrix base comprises collagen.

16. The method of claim 14, wherein the extracellular matrix base comprises rat-tail collagen.

17. The method of any one of claims 1 through 16, wherein the intestinal epithelial cells are nontumorigenic.

18. The method of any one of claims 1 through 17, wherein the intestinal epithelial cells are human.

19. The method of any one of claims 1 through 17, wherein the intestinal epithelial cells are derived from a human duodenal biopsy.

20. The method of any one of claims 1 through 19, wherein the intestinal epithelial cells are derived from SCBN cells.

21. The method of any one of claims 1 through 20, wherein the transepithelial electrical resistance of the confluent cell layer is less than about 200 ohm.cm².

22. The method of any one of claims 1 through 20, wherein the transepithelial electrical resistance of the confluent cell layer is less than about 150 ohm.cm².

23. The method of any one of claims 1 through 20, wherein the transepithelial electrical resistance of the confluent cell layer is less than about 100 ohm.cm².

24. The method of any one of claims 1 through 23, wherein greater than 5% of the intestinal epithelial cells have microvilli longer than 1 micrometer.

25. The method of any one of claims 1 through 23, wherein greater than 10% of the intestinal epithelial cells have microvilli longer than 1 micrometer.

26. The method of any one of claims 1 through 23, wherein greater than 15% of the intestinal epithelial cells have microvilli longer than 1 micrometer.

27. The method of any one of claims 1 through 23, wherein greater than 20% of the intestinal epithelial cells have microvilli longer than 1 micrometer.

28. The method of any one of claims 1 through 23, wherein greater than 25% of the intestinal epithelial cells have microvilli longer than 1 micrometer.

29. The method of any one of claims 1 through 23, wherein greater than 30% of the intestinal epithelial cells have microvilli longer than 1 micrometer.

30. The method of any one of claims 1 through 29, wherein the culture media is essentially free of animal serum.

31. The method of any one of claims 1 through 29, wherein the culture media comprises D-glucose, L-glutamine, sucrose, pyruvic acid, a pH buffer and one or more amino acids.

32. The method of any one of claims 1 through 31, wherein the culture is maintained at a temperature of about 36 degrees Celsius to 38 degrees Celsius.

33. An in vitro culture comprising a confluent layer of intestinal epithelial cells, wherein:
a) the cells express an enzyme activity selected from the group consisting of alkaline phosphatase, dipeptidyl peptidase IV, aminopeptidase and amino peptidase A;
b) the confluent layer has a transepithelial electrical resistance less than about 200 ohm.cm²; and
c) greater than 5% of the cells have microvilli longer than 1 micrometer.

34. The culture of claim 33, wherein the culture is a long-term culture having the enzyme activity, transepithelial electrical resistance and microvilli of claim 33 over a period of at least 48 hours starting about 72 hours after seeding the plates.

35. The culture of claim 33 or 34, wherein the confluent layer is grown on an extracellular matrix base.

36. The culture of claim 35, wherein the extracellular matrix base comprises collagen.

37. The culture of claim 35, wherein the extracellular matrix base comprises rat-tail collagen.

38. The culture of any one of claims 33 through 37, wherein the intestinal epithelial cells are nontumorigenic.

39. The culture of any one of claims 33 through 38, wherein the intestinal epithelial cells are human.

40. The culture of any one of claims 33 through 39, wherein the intestinal epithelial cells are derived from a human duodenal biopsy.

41. The culture of any one of claims 33 through 40, wherein the intestinal epithelial cells are derived from SCBN cells.

42. The culture of any one of claims 33 through 41, wherein greater than 5% of the intestinal epithelial cells have microvilli longer than 1 micrometer.

43. The culture of any one of claims 33 through 41, wherein greater than 10% of the intestinal epithelial cells have microvilli longer than 1 micrometer.

44. The culture of any one of claims 33 through 41, wherein greater than 15% of the intestinal epithelial cells have microvilli longer than 1 micrometer.

45. The culture of any one of claims 33 through 41, wherein greater than 20% of the intestinal epithelial cells have microvilli longer than 1 micrometer.

46. The culture of any one of claims 33 through 41, wherein greater than 25% of the intestinal epithelial cells have microvilli longer than 1 micrometer.

47. The culture of any one of claims 33 through 41, wherein greater than 30% of the intestinal epithelial cells have microvilli longer than 1 micrometer.

48. A cell culture media capable of supporting an in vitro culture comprising a confluent layer of intestinal epithelial cells, wherein:
a) the cells express an enzyme activity selected from the group consisting of alkaline phosphatase, dipeptidyl peptidase IV, aminopeptidase and amino peptidase A;
b) the confluent layer has a transepithelial electrical resistance less than about 200 ohm.cm²; and
c) greater than 5% of the cells have microvilli longer than 1 micrometer.

49. A cell culture media comprising nutrients effective to support the viability of mammalian cells, and further comprising sodium butyrate and 2-{1-[3-(amidinothio)propyl]-1H-indol-3-yl}-3-(1-meethylindol-3-yl)-maleimide methanesulfonate.

50. The cell culture media of claim 49, wherein the effective amount of the kinase modulator is greater than 1 micromolar.

51. The cell culture media of claim 49, wherein the effective amount of the kinase modulator is from about 1 micromolar to about 10 micromolar.

52. The cell culture media of claim 49, wherein the effective amount of the kinase modulator is about 5 micromolar.

53. The cell culture media of any one of claims 49 through 52, wherein the effective amount of the differentiating compound is greater than 1 mM.

54. The cell culture media of any one of claims 49 through 52, wherein the effective amount of the differentiating compound is from about 1 mM to about 10 mM.

55. The cell culture media of any one of claims 49 through 52, wherein the effective amount of the differentiating compound is about 5 mM.

56. The cell culture media of any one of claims 49 through 55, wherein the culture media is essentially free of animal serum.

57. The cell culture media of any one of claims 49 through 56, wherein the culture media comprises D-glucose, L-glutamine, sucrose, pyruvic acid a pH buffer and one or more amino acids.

58. A method for determining if a culture media is capable of supporting an in vitro culture comprising a confluent layer of intestinal epithelial cells, wherein: a) the cells express an enzyme activity selected from the group consisting of alkaline phosphatase, dipeptidyl peptidase IV, aminopeptidase and amino peptidase A; b) the confluent layer has a transepithelial electrical resistance less than about 200 ohm.cm²; and c) greater than 5% of the cells have microvilli longer than 1 micrometer, comprising selecting a possible candidate media, contacting said media with SCBN and measuring the SCBN growth in said media to determine if a) the cells express an enzyme activity selected from the group consisting of alkaline phosphatase, dipeptidyl peptidase IV, aminopeptidase and amino peptidase A; b) the confluent layer has a transepithelial electrical resistance less than about 200 ohm.cm²; and, c) greater than 5% of the cells have microvilli longer than 1 micrometer.

59. The culture media determined by the method of claim 58 if not previously known.

60. A composition comprising an extract of the cell culture of any of claims 33-42.

61. The composition of claim 60, wherein the extract is an RNA extract.

62. A method for determining genes which control the expression of enzyme activity in a cell, wherein a) the enzyme activity is selected from the group consisting of alkaline phosphatase, dipeptidyl peptidase IV, aminopeptidase and amino peptidaseA;
b) the cell has a transepithelial electrical resistance less than about 200 ohm.cm²; and,
c) when the said cells are grown in a culture comprising the following steps, greater than 5% of the cells have microvilli longer than 1 micrometer:
i) growing the SimBioDAS® cells in a culture; and
ii) characterizing the genes expressed in said culture to determine which genes control the above expression,

63. The genes determined by the method of claim 62 if they are not previously known.

64. A composition comprising the genes determined by the method of claim 62 in an effective concentration capable of transforming appropriate cells to control the expression of enzyme activity in a cell, wherein a) the enzyme activity is selected from the group consisting of alkaline phosphatase, dipeptidyl peptidase IV, aminopeptidase and amino peptidase A; b) the cell has a transepithelial electrical resistance less than about 200 ohm.cm²; and c) when the said cells are grown in a culture, greater than 5% of the cells have microvilli longer than 1 micrometer.

65. A method for preparing the cell culture of claim 33-42 comprising introducing the genes capable of controlling the expression of an enzyme activity in a cell, wherein a) the enzyme activity is selected from the group consisting of alkaline phosphatase, dipeptidyl peptidase IV, aminopeptidase and amino peptidase A; b) the cell has a transepithelial electrical resistance less than about 200 ohm.cm²; and c) when the said cells are grown in a culture, greater than 5% of the cells have microvilli longer than 1 micrometer.

66. The method of claim 65, wherein the genes are recited in Table 2.

67. The cell lines derived from the cell culture prepared by the method of claim 64.

68. A method of processing an aqueous composition comprising a plurality of compounds to obtain a physiologically modified composition, the method comprising: applying the aqueous composition to a first side of an in vitro confluent intestinal epithelial cell layer; and, extracting the modified composition from a second side of the confluent intestinal epithelial cell layer; wherein:
a) cells of the confluent intestinal epithelial cell layer express an enzyme activity selected from the group consisting of alkaline phosphatase, dipeptidyl peptidase IV, aminopeptidase and amino peptidase A;
b) the confluent intestinal epithelial cell layer has a transepithelial electrical resistance less than about 200 ohm.cm²; and,
c) a portion of the cells of the confluent intestinal epithelial cell layer have microvilli longer than 1 micrometer.

69. The method of claim 68, wherein cells of the confluent intestinal epithelial cell layer are nontumorigenic.

70. The method of claim 68 or 69, wherein cells of the confluent intestinal epithelial cell layer are human.

71. The method of any one of claims 68 through 70, wherein cells of the confluent intestinal epithelial cell layer are derived from a human duodenal biopsy.

72. The method of any one of claims 68 through 71, wherein cells of the confluent intestinal epithelial cell layer are derived from SCBN cells.

73. The method of any one of claims 68 through 72, wherein the transepithelial electrical resistance of the confluent cell layer is less than about 200 ohm.cm².

74. The method of any one of claims 68 through 73, wherein greater than 5% of the intestinal epithelial cells have microvilli longer than 1 micrometer.

75. The method of any one of claims 68 through 74, wherein the aqueous composition comprising a plurality of compounds is a plant extract.

76. The method of any one of claims 68 through 75, wherein the confluent intestinal epithelial cell layer is grown in a culture media comprising:
a) an amount of a differentiating compound effective to maintain the transepithelial electrical resistance of the confluent cell layer; and, b) an amount of a kinase modulator effective in combination with the differentiating compound to support growth of microvilli longer than 1 micrometer on the apical portion of the cells.

77. A physiologically modified composition produced by the method of any one of claims 68 through 76.

78. A method of treating intestinal epithelial cells comprising administering to the cells a differentiating compound in combination with an amount of 2-{1-[3-(amidinothio)propyl]-1H-indol-3-yl}-3-(1-meethylindol-3-yl)-maleimide methanesulfonate effective to support growth of microvilli longer than 1 micrometer on a portion of the epithelial cells.

79. An epithelial cell culture substantially as hereinbefore described and with reference to the examples and drawings.

80. A ginkgo extract processed according to the method of any one of claims 68 through 76.

81. A ginkgo extract substantially as hereinbefore described and with reference to the examples and drawings.

82. A profile generated by the physiologically modified composition of claim 77.

83. The profile of claim 82 indicating that the composition is therapeutically useful.

84. A method to determine the batch variation of a natural product comprising steps of:
a) generating a profile of claim 83; and
b) comparing the profile of each batch of the product to determine whether said batch would be therapeutically useful.

85. A method to identify needed absorbable active ingredients by further purification of the ingredients identified in the profile of claim 82.

86. The absorbable active ingredient identified by the method of claim 85.

87. A composition comprising the absorbable active ingredient of claim 86.

88. A pharmaceutical composition comprising the absorbable active ingredient of claim 86 and a pharmaceutically acceptable carrier.

89. A method for treating a subject comprising administering an effective amount of the pharmaceutical composition of claim 88 to the subject.

90. The gene expression profile derived from the microarray study using the SimBioDAS® cell.

91. The gene expression profile of claim 90, used to identify the cell signaling pathway involved in SimBioDAS® cell differentiation.

92. The signaling pathway identified by claim 91 if it is not previously known.

93. A method for manipulation of the cell signaling pathways of SimBioDAS® cells through genetic manipulation or application of kinase inhibitors/activators in the tissue culture medium to achieve fuller cell differentiation by the information of the gene expression profile of claim 91 or the signaling pathway of claim 92.

94. The manipulated cell of claim 91.

95. The extract of the manipulated cell of claim 91.

96. A chemical compound screening method of processing an aqueous composition comprising a solution containing a chemical drug candidate compound to obtain a physiologically modified composition, the method comprising: applying the aqueous composition to a first side of an in vitro confluent intestinal epithelial cell layer; and, extracting the modified composition from a second side of the confluent intestinal epithelial cell layer; wherein:
a) cells of the confluent intestinal epithelial cell layer express an enzyme activity selected from the group consisting of alkaline phosphatase, dipeptidyl peptidase IV, aminopeptidase and amino peptidase A;
b) the confluent intestinal epithelial cell layer has a transepithelial electrical resistance less than about 200 ohm.cm²; and,
c) a portion of the cells of the confluent intestinal epithelial cell layer have microvilli longer than 1 micrometer.

97. The method of claim 94, wherein cells of the confluent intestinal epithelial cell layer are nontumorigenic.

98. The method of claim 96 or 97, wherein cells of the confluent intestinal epithelial cell layer are human.

99. The method of any one of claims 96 through 98, wherein cells of the confluent intestinal epithelial cell layer are derived from a human duodenal biopsy.

100. The method of any one of claims 96 through 99, wherein cells of the confluent intestinal epithelial cell layer are derived from SCBN cells.

101. The method of any one of claims 96 through 100, wherein the transepithelial electrical resistance of the confluent cell layer is less than about 200 ohm.cm².

102. The method of any one of claims 96 through 101, wherein greater than 5% of the intestinal epithelial cells have microvilli longer than 1 micrometer.

103. The method of any one of claims 96 through 102, wherein the aqueous composition comprising a solution of a compound is a chemical compound being screened as a drug candidate.

104. The method of any one of claims 96 through 103, wherein the confluent intestinal epithelial cell layer is grown in a culture media comprising:
a) an amount of a differentiating compound effective to maintain the transepithelial electrical resistance of the confluent cell layer; and,
b) an amount of a kinase modulator effective in combination with the differentiating compound to support growth of microvilli longer than 1 micrometer on the apical portion of the cells.

105. A physiologically modified composition produced by the method of any one of claims 96 through 104.
